(19) United States
(12) Patent Application Publication
(10) Pub. No.: US 2025/0269023 A1
(43) Pub. Date: Aug. 28, 2025

(54) NOVEL THERAPIES WITH ENGINEERED EFFECTOR CELLS

(71) Applicant: THE SCRIPPS RESEARCH INSTITUTE, La Jolla, CA (US)

(72) Inventors: Travis Young, La Jolla, CA (US); Nelson Bruno, San Diego, CA (US)

(21) Appl. No.: 18/554,340

(22) PCT Filed: Apr. 7, 2022
(86) PCT No.: PCT/US22/23791

§ 371 Date: Jan. 31, 2024

Related U.S. Application Data
(60) Provisional application No. 63/172,212, filed on Apr. 8, 2021.

(51) Int. Cl. A61K 40/42; A61K 40/11; A61K 40/31; C07K 14/725; C07K 16/28

(52) U.S. Cl. CPC A61K 40/4211; A61K 40/11; A61K 40/31; C07K 14/7051; C07K 16/2803; C07K 2317/622

(57) ABSTRACT

The invention novel methods for engrafting or expanding a switchable engineered effector cell (e.g., CAR-T cell) in a subject who has not undergone lymphodepleting (LD) chemotherapy. The methods entail administering to a subject a switchable effector platform (e.g., sCAR-T platform). The platform includes a switch molecule that specifically binds to a target molecule on the surface of a cell in the subject, and a complementary engineered effector cell (e.g., CAR-T cell) containing a CAR extracellular domain that specifically binds to a CAR-interacting domain in the switch molecule. Some methods of the invention are directed to treating or promoting regression of tumors in subjects who have not undergone lymphodepleting (LD) chemotherapy.

### CROSS-REFERENCE TO RELATED APPLICATIONS

The subject patent application claims the benefit of priority to U.S. Provisional Patent Application No. 63/172,212 (filed Apr. 8, 2021; now pending). The full disclosure of the priority application is incorporated herein by reference in its entirety and for all purposes.

### STATEMENT OF GOVERNMENT SUPPORT

This invention was made with government support under grant number CA208398 awarded by the National Institutes of Health. The government has certain rights in the invention.

### SEQUENCE LISTING

This application incorporates by reference a Sequence Listing in the form of a ST.26 XML file labeled “SeqListing_ST25”. The file is of 14 KB and was created on Jan. 23, 2024.

### BACKGROUND OF THE INVENTION

CAR-T therapies involve adoptive transfer of genetically modified T cells to “reteach” the immune system to recognize and eliminate malignant tumor cells. Conventional CAR-T treatment platform requires high cells doses and also non-myeloablative lymphodepleting (LD) chemotherapy preconditioning. For example, adoptive transfer of 10⁵-10⁶ CAR-T cells per mouse is common in mouse models. Human doses are typically in the range of 1-2×10⁶ cells/kg (see Kyrmriah and Yescarta product inserts). LD is a standard practice in the field for both mouse models and human studies (see Kyrmriah and Yescarta product inserts). LD preconditioning has been shown to be required in CAR-T cell therapy prior to adoptive transfer of the cells for three key reason (a) the creation of space in the secondary lymph for homeostatic proliferation of the adoptively transferred CAR-T cells, (b) the elimination of cytokine “sinks”/reduction of competition for cytokines, (c) the elimination of suppressive cell populations such as Tregs.

There is a need in the art for better and more cost-effective immunotherapies for treating cancers. The instant invention is directed to addressing these and other unmet needs.

### SUMMARY OF THE INVENTION

In one aspect, the invention provides methods for engrafting or expanding an engineered T cell in a subject in need thereof. These methods involve contacting the subject with (a) a chimeric antigen receptor-T cell switch molecule (CAR-T switch) that contains (i) a chimeric antigen receptor-interacting domain (CAR-ID) and (ii) a targeting moiety, and (b) a complementary CAR-T cell having in the extracellular domain of its CAR a single-chain variable fragment (scFv) that specifically binds to the CAR-ID. Typically, the subject has not undergone lymphodepletion (LD) chemotherapy prior to being administered the CAR-T switch and the CAR-T cells. In various embodiments, the targeting moiety in the switch molecule specifically binds to a cell surface target molecule in the subject. In some embodiments, the target molecule is present on the surface of a cell associated with or implicated in a disease in the subject. In some of these embodiments, the disease is a cancer or an autoimmune disease. In some embodiments, the targeting moiety contains an antibody moiety or a small organic molecule moiety that specifically binds the target molecule.

Some methods of the invention are specifically directed to treating, arresting growth of, and/or promoting regression of, a cancer in a subject that has not undergone lymphodepletion (LD) chemotherapy. These methods entail administering to the subject (a) a first chimeric antigen receptor-T cell switch molecule (CAR-T switch) that contains (i) a chimeric antigen receptor-interacting domain (CAR-ID) and (ii) a targeting moiety containing an antibody or antigen-binding fragment thereof, and (b) a complementary CAR-T cell containing in the extracellular domain of its CAR a single-chain variable fragment (scFv) that specifically binds to the CAR-ID. The targeting moiety of the CAR-T switch is capable of specifically binding to a cell surface molecule of the cancer in the subject. Preferably, the subject to be treated is a human.

In some embodiments, the subject is administered with one dose of the CAR-T cell at the beginning of the treatment, and multiple doses of the CAR-T switch during the course of the treatment. In some embodiments, a single dose of the CAR-T cells administered to the subject is less than about 1×10⁵ cells per kg of body weight. In various embodiments, a single dose of CAR-T cells administered to the subject is less than about 7.5×10⁴, 5×10⁴, 2.5×10⁴, 1×10⁴, 7.5×10³, 5×10³, 2.5×10³, 1×10³, 750, or 500 cells per kg of body weight. In some embodiments, a single dose of the CAR-T switch administered to the subject is from about 0.01 mg to about 1 mg per kg of body weight.

In some embodiments, the CAR-ID of the administered CAR-T switch contains a peptide or a small molecule. In some of these embodiments, the peptide is a GCN4 derivative peptide, and the CAR extracellular domain contains an anti-GCN4 scFv. In some embodiments, the GCN4 derivative peptide contains the amino acid sequence set forth in SEQ ID NO:1, a substantially identical sequence or a conservatively modified variant thereof. In some embodiments, wherein the anti-GCN4 scFv contains a light chain variable region sequence set forth in SEQ ID NO:4, and a heavy chain variable region sequence set forth in SEQ ID NO:5. In some of these embodiments, the anti-GCN4 scFv contains the amino acid sequence set forth in SEQ ID NO:7. In some embodiments, the small molecule in the CAR-ID is fluorescein isothiocyanate (FITC), and the CAR extracellular domain contains an anti-FITC scFv.

In various embodiments, the targeting moiety of the administered CAR-T switch is an antibody or antigen-binding fragment thereof that specifically binds to a cell surface molecule selected from the group consisting of CD19, CD20, CD22, CD33, CEA, CLL1, BCMA, CS1, CD123 and Her2. In some embodiments, the targeting moiety is an anti-CD19 antibody or antigen-binding fragment thereof that contains a light chain variable region and a heavy chain variable region sequence set forth as SEQ ID NOs: 2 and 3, respectively.

In some methods of the invention, the subject to be treated can be further administered a second CAR-T switch molecule containing a targeting moiety that specifically binds to a second cell surface molecule of the cancer in the subject. In some of these embodiments, the second CAR-T switch molecule can contain a chimeric antigen receptor-interacting domain (CAR-ID) that is specifically recognized by the scFv in the CAR of the complementary CAR-T cell. In some of these embodiments, the first CAR-T switch molecule contains an anti-CD19 antibody or antigen binding fragment thereof, and the second CAR-T switch molecule contains an anti-CD20 antibody or antigen binding fragment thereof.

In various embodiments, the CAR-T switch and the complementary CAR-T cells can be administered to the subject either simultaneously or sequentially. In some embodiments, the CAR of the administered CAR-T cell is humanized and contains an amino acid sequence set forth in SEQ ID NO:6. In some embodiments, the cancer to be treated is a heterogeneous tumor or a blood cell malignancy. In some embodiments, the cancer to be treated is a CD19-positive malignancy. In various embodiments, the CD19-positive malignancy to be treated is acute lymphoblastic leukemia, acute myeloid leukemia, or chronic lymphocytic leukemia. In some embodiments, the CD19-positive malignancy to be treated is a CD19-positive B cell cancer. In some of these embodiments, the CD19-positive B cell cancer to be treated can be selected from the group consisting of diffuse large B-cell lymphoma (DLBCL), follicular lymphoma, chronic lymphocytic leukemia (CLL), small lymphocytic lymphoma (SLL), mantle cell lymphoma (MCL), marginal zone lymphoma, Burkitt lymphoma, lymphoplasmacytic lymphoma (Waldenstrom macroglobulinemia), hairy cell leukemia (HCL), primary central nervous system (CNS) lymphoma, and primary intraocular lymphoma.

A further understanding of the nature and advantages of the present invention may be realized by reference to the remaining portions of the specification and claims.

### DESCRIPTION OF THE DRAWINGS

FIG. 1 shows anti-CD19 targeting sCAR-T treatment in mouse model. (A) Scheme of treatment with the sCAR-T platform comprised of anti-murine CD19 switch and murine sCAR-T cells in a syngeneic mouse model with or without LD preconditioning. (B) Results showing B cells from peripheral blood of animals treated with the anti-CD19 targeting sCAR-T platform.

FIG. 2 shows additional results of treatment of syngeneic mouse model with anti-CD19 targeting sCAR-T platform. (A) Percentage of CD3⁺ CAR-T cells in circulation, indicating that the on/off cycling in the administration of the CAR-T switch to be a key for in vivo CAR-T cell expansion. (B) Progressive accumulation of CD44⁺/CD62L⁺ (memory) sCAR-T cells.

FIG. 3 shows the sCAR-T treatment scheme and results from titration studies to determine the lower limit of sCAR-T cell engraftment. Mice treated with a single dose of sCAR-T cells at 1×10⁶ or a tenfold lower dose at 1×10⁵ cells/mouse, showed an almost total depletion of B cells in blood.

FIG. 4 shows results from titration studies to determine the lower limit of sCAR-T cell engraftment. (A) With both 1×10⁶ and 1×10⁵ doses of sCAR-T cells, there were cells detectable by flow cytometry. (B) Progressive accumulation CD44⁺/CD62L⁺ (memory) sCAR-T over time.

FIG. 5 shows the sCAR-T treatment scheme and structure of CAR used in 1D3 conventional CAR platform for comparison studies.

FIG. 6 shows results from a comparison of treatment of mouse tumor model with the sCAR-T platform and a conventional CAR-T platform. (A) CD19⁺ cell population was unaffected in mice treated with conventional CAR-T platform, while a near complete depletion of CD19⁺ cells was observed in the sCAR-T treated mouse group at a dose of 3×10⁵ cells. (B) Percentage of CD3⁺ CAR-T cells in blood in the differently treated mouse groups.

### DETAILED DESCRIPTION

### I. Overview

The invention is predicated in part on the studies undertaken by the present inventors to challenge existing paradigms of conventional CAR-T therapies. Utilizing a switchable CAR-T (sCAR-T) platform, the inventors demonstrated that LD is not required for the therapeutic activities of the sCAR-T cells in vivo in mouse tumor models. Results from this study are unexpected as it has been documented in the literature that LD is required for CAR-T therapies in mouse models. See, e.g., Davila et al., PLOS ONE 8 (4): e61338, 2013. Not intended to be bound by the theory, it is believed that such improvements are enabled by the switch dosing and cannot be achieved with conventional CAR-T cell designs. sCAR-T cell expansion from very low numbers in the absence of preconditioning is likely due to the cyclical (or pulsatile) stimulation from the switch, which allows “rest” periods between activation states. This is different from the conventional CAR-T therapies wherein the cells are always on (i.e., constitutively firing). The constitutive firing of conventional CAR-T cells is expected to lead to exhaustion, while the cyclical or pulsatile activation of the sCAR-T cell does not.

In accordance with these studies, the present invention provides methods of engrafting engineered effector cells (e.g., CAR-T cells) in subjects who are in need of treatment of the cell but who have not previously received LD preconditioning prior to the grafting. As exemplification, the invention provides novel cancer therapies with the sCAR-T platforms described herein. Specifically, the invention enables CAR-T cancer therapies for treating human cancer patients without the need to first undergo LD preconditioning. Methods described herein are also suitable for engrafting of other engineered effector cells and corresponding targeting modules or switch molecules that are known in the art. In addition to applications for cancer therapies, methods of the invention can also be employed in the treatment of various other diseases or conditions. These include, e.g., autoimmune diseases and infections (viral or fungal infections).

Unless otherwise stated, the present invention can be performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13:978-0121821906); U.S. Pat. Nos. 4,965,343, and 5,849,954; Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2nd ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998).

The following sections provide additional guidance for practicing the compositions and methods of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

The term “antibody” typically refers to polypeptide chain(s) which exhibit a strong monovalent, bivalent or polyvalent binding to a given antigen, epitope or epitopes. Unless otherwise noted, antibodies or antibody fragments used in the invention also encompass certain immunoglobulin molecules or variant antibodies that do not possess specific antigen-binding activities, e.g., catalytic antibodies. immunoglobulin s can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antibody fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4} Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

As used herein, an antibody fragment (or antigen binding fragment of an antibody) refers to any proteins or polypeptides that contain at least one antibody-derived V_{H}, V_{L}, or C_{H} immunoglobulin domain in the context of other non-immunoglobulin, or non-antibody derived components. Such molecules include, but are not limited to (i) F_{c}-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

“Humanized” forms of non-human (e.g., rodent, e.g., murine or rabbit) immunoglobulins are immunoglobulins which contain minimal sequences derived from non-human immunoglobulin. For the most part, humanized immunoglobulins are human immunoglobulins (recipient antibody) in which residues from a hypervariable region of the recipient are replaced by residues from a hypervariable region of a non-human species (donor antibody) such as mouse, rat, hamster, rabbit, chicken, bovine or non-human primate having the desired specificity, affinity, and capacity. In some instances, Fv framework region (FR) residues of the human immunoglobulin are also replaced by corresponding non-human residues. Furthermore, humanized antibodies can comprise residues which are not found in the recipient antibody or in the donor antibody. These modifications are made to further refine antibody performance. In general, the humanized immunoglobulin will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized immunoglobulin optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

The term “human immunoglobulin”, as used herein, is intended to include immunoglobulins having variable and constant regions derived from human germline immunoglobulin sequences. The human immunoglobulins of the invention can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human immunoglobulin”, as used herein, is not intended to include immunoglobulins in which CDR sequences derived from the germline of another mammalian species, such as a mouse, have been grafted onto human framework sequences.

Lymphodepleting (LD) chemotherapy or lymphodepleting preconditioning refers to a therapy administered to subjects before CAR-T cell infusion, in order to deplete endogenous T-cells (and Tregs), so that they are not going to antagonize/suppress and allow expansion/proliferation of the infused CAR-T cells. One example of LD preconditioning is to administer the subjects with Fludarabine+Cyclophosphamide (FluCy).

The term “specific binding” or “specifically binds to” or is “specific for” refers to the binding of a binding moiety to a binding target, such as the binding of an immunoglobulin or small molecule agent to a target molecule or antigen, e.g., an epitope on a particular polypeptide, peptide, or other target (e.g. a glycoprotein target), and means binding that is measurably different from a non-specific interaction (e.g., a non-specific interaction can be binding to bovine serum albumin or casein). Specific binding can be measured, for example, by determining binding of a binding moiety (e.g., a small molecule agent), or an immunoglobulin, to a target molecule compared to binding to a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “specific binding” or “specifically binds to” or is “specific for” a particular target molecule or an epitope on a particular target molecule can be exhibited, for example, by a molecule having a K_{d} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain instances, the term “specific binding” refers to binding where a binding moiety binds to a particular target molecule or epitope on the target molecule without substantially binding to any other molecule or epitope.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini. The term “fusion” is used herein to refer to the combination of amino acid sequences of different origin.

The term “epitope” includes any molecular determinant capable of specific binding to an immunoglobulin. In certain aspects, epitope determinants include chemically active surface groupings of molecules such as amino acids, sugar side chains, phosphoryl, or sulfonyl, and, in certain aspects, can have specific three dimensional structural characteristics, and/or specific charge characteristics. An epitope is a region of an antigen that is bound by an immunoglobulin. A “binding region” is a region on a binding target bound by a binding molecule.

The term “target” or “binding target” is used in the broadest sense and specifically includes polypeptides, without limitation, nucleic acids, carbohydrates, lipids, cells, and other molecules with or without biological function as they exist in nature. In some specific embodiments, the term target refers to a cell surface molecule on a target cell, e.g., a tumor cell.

The term “antigen” refers to an entity or fragment thereof, which can bind to an immunoglobulin or trigger a cellular immune response. An immunogen refers to an antigen, which can elicit an immune response in an organism, particularly an animal, more particularly a mammal including a human. The term antigen includes regions known as antigenic determinants or epitopes, as defined above.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to a peptide or polypeptide sequence, i.e., an scFV antibody polypeptide sequence or a GCN4 derived peptide identified herein, is defined as the percentage of amino acid residues in a candidate sequence that are identical with the amino acid residues in the specific peptide or polypeptide sequence after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Two sequences are “substantially identical” if they have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the well-known sequence comparison algorithms or by manual alignment and visual inspection.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for cancer, if, after receiving a treatment of the present invention, the subject shows observable and/or measurable reduction in or absence of one or more of the following: reduction in the number of cancer cells or absence of the cancer cells; reduction in the tumor size; inhibition (i.e., slowing to some extent and preferably stopping) of cancer cell infiltration into peripheral organs, including the spread of cancer into soft tissue and bone; inhibition (i.e., slowing to some extent and preferably stopping) of tumor metastasis; inhibition, to some extent, of tumor growth; and/or relief to some extent of one or more of the symptoms associated with the specific cancer; reduced morbidity and/or mortality, and improvement in quality of life issues.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

The term “subject” refers to human and non-human animals (especially non-human mammals). The term “subject” is used herein, for example, in connection with the disclosed therapeutic methods, to refer to human or non-human animals. Specific examples of non-human subjects include, e.g., cows, horses, sheep, pigs, cats, dogs, mice, rats, rabbits, guinea pigs, monkeys.

Artificial T cell receptors (also known as chimeric T cell receptors, chimeric immunoreceptors, chimeric antigen receptors (CARs) or T-bodies) are engineered receptors, which graft an arbitrary specificity onto an immune effector cell. Typically, these receptors are used to graft the specificity of a monoclonal antibody onto a T cell; with transfer of their coding sequence facilitated by retroviral or lentiviral vectors or by transposons. CAR-engineered T cells (also abbreviated CAR-T cells) are genetically engineered T cells armed with chimeric receptors whose extracellular recognition unit is comprised of an antibody-derived recognition domain and whose intracellular region is derived from one or more lymphocyte stimulating moieties. The structure of the prototypic CAR is modular, designed to accommodate various functional domains and thereby to enable choice of specificity and controlled activation of T cells. The preferred antibody-derived recognition unit is a single chain variable fragment (scFv) that combines the specificity and binding residues of both the heavy and light chain variable regions of a monoclonal antibody. The most common lymphocyte activation moieties include a T-cell costimulatory (e.g. CD28 and/or 4-1BB) domain in tandem with a T-cell triggering (e.g. CD3zeta) moiety. By arming effector lymphocytes (such as T cells and natural killer cells) with such chimeric receptors, the engineered cell is re-directed with a pre-defined specificity to any desired target antigen, in a non-HLA restricted manner. CAR constructs are introduced ex vivo into T cells from peripheral lymphocytes of a given patient using retroviral or lentiviral vectors or transposons. Following infusion of the resulting CAR-engineered T cells back into the patient, they traffic, reach their target site, and upon interaction with their target cell or tissue, they undergo activation and perform their predefined effector function. Therapeutic targets for the CAR approach include cancer and HIV-infected cells, or autoimmune effector cells.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

A sCAR-T platform refers to a CAR-T switch molecule and a complementary CAR-T cell. The CAR-T switch molecule (“CAR-T switch”) contains a targeting moiety (e.g., an antibody or antigen-binding fragment thereof) that is capable of specifically binding to a target molecule on the surface of a target cell (e.g., a tumor cell). The CAR-T switch is also able to bind to the CAR of the complementary CAR-T cell. Typically, the extracellular domain of the CAR of the CAR-T cell contains an antibody moiety (e.g., a scFv) that specifically recognizes a CAR-ID domain (e.g., a peptide or a small molecule) in the CAR-T switch.

### III. Novel CAR-T Therapies not Requiring Lymphodepleting Preconditioning

In conventional CAR-T therapies, subjects to be treated need to be preconditioned with lymphodepleting chemotherapy (aka “LD preconditioning” or “LD conditioning”). This LD preconditioning is an essential step before the subjects can be actually infused with the CAR-T cells. It creates a “favorable” environment for CAR T-cell expansion and survival in vivo, presumably by eliminating regulatory T cells. LD preconditioning can lead to the upregulation of tumor immunogenicity and improve disease control. It has been shown that LD preconditioning works to promote homeostatic proliferation of adoptively transferred T cells via increases in the pro-survival/proliferation cytokines, interleukin (IL)-7 and IL-15, and in conjunction with a lack of competition with wildtype T cells. See, e.g., Hirayama et al., Blood 133:1876-1887, 2019; Hay and Turtle, Drugs 77:237-245, 2017; and Yakoub-Agha et al., Haematologica. 105:297-316, 2020.

In one aspect, the invention provides methods for engrafting or expanding an engineered effector lymphocyte (e.g., CAR-T cells) in a subject. Typically, the subject is one afflicted with a disease or condition (e.g., a cancer) that the engineered effector cells are intended to treat. In addition, the subject has not undergone LD chemotherapy prior to receiving the cells for expansion. In these methods, the subject is administered with (a) a switch molecule or targeting module that turns on the effector cell that contains (i) a chimeric antigen receptor-interacting domain (CAR-ID) and (ii) a targeting moiety that is specific for a molecule manifesting the disease or condition afflicted by the subject (e.g., a tumor cell surface molecule), and (b) a complementary engineered effector cell (e.g., CAR-T cell) that has in the extracellular domain of its CAR a single-chain variable fragment (scFv) that specifically binds to the CAR-ID.

In one aspect, the invention provides novel therapies with engineered effector cells (e.g., CAR-T therapies) for treating or ameliorating the symptoms of various diseases or conditions in needing subjects (in particular, human patients). Diseases suitable for methods of the invention include various cancers, tumors or malignancies, as well as some other diseases or disorders such as autoimmune diseases and infections. Some embodiments of the invention are particularly directed to treating cancers. In some related embodiments, the novel therapies of the invention are directed to promoting tumor regression and/or to arresting tumor growth in subjects afflicted with various malignancies. In general, the subjects to be treated with the therapies of the invention do not need to receive, and have not previously undergone, LD chemotherapy. Specifically, subjects who have not previously undergone LD chemotherapy are administered a switchable CAR-T cell platform (sCAR-T) that is designed for the treatment of a specific cancer that the subject is afflicted with. The switchable CAR-T cell platform contains (a) a CAR-T switch (also referred to herein as CAR-T switch molecule, including CAR-T switch polypeptide or CAR-T switch compound) that can bind to the CAR of a CAR-T cell and also specifically targets a cell surface molecule on a tumor cell, and (b) a complementary CAR-T cell that contains a CAR that can be bound by the switch. When treating human subjects, the administered CAR-T switch and the complementary CAR-T cells are preferably human or humanized. For example, the CAR can be a humanized polypeptide, and the T cell in which the CAR is to be expressed can be human cells. In some of these embodiments, the T cells expressing the humanized CAR are autologous T cells isolated from the specific human subject to be treated. In general, the administration can be performed in accordance with standard protocols of immunotherapy or the more detailed guidance provided herein. In some preferred embodiments, the CAR-T switch and the complementary CAR-T cells are administered to the subject by infusion. In some embodiments, the sCAR-T treatment methods described herein can be used in combination with other known therapies or therapeutic agents for treating cancers, e.g., chemotherapy, hormone therapy, radiation therapy, or surgery.

In some methods, the subject to be treated can be administered with more than one CAR-T switches, along with the complementary CAR-T cells, that target different surface molecules on a tumor cell. In some preferred embodiments, the different CAR-T switch molecules contain the same CAR-ID domain, which allows the different switches to interact with the same complementary CAR-T cell. Such a treatment is especially beneficial for heterogeneous tumors. For example, subjects afflicted with leukemias and lymphomas can be treated with a pharmaceutical composition that contains (a) both a CD19-targeting CATR-T switch plus a CD20- or CD22-targeting switch, and (b) the complementary CAR-T cells. In these embodiments, the CAR-T switches can be administered sequentially or simultaneously. A second switch targeting a second cell surface molecule on the target cell may be administered after down regulation of a first cell surface molecule on the target cell that is targeted by a first switch.

Methods described herein are also suitable for engrafting of other engineered effector cells and corresponding targeting modules (“switches”) that are known in the art. For example, methods of engineered T cell engrafting without LD preconditioning can be used with the UniCAR-T, RevCAR-T and ddBCMA-CAR-T platforms. See, e.g., Loff et al., Molecular Therapy Oncolytics 2020; 17:408; Wermke et al., Blood 2021; 137:3145-3148; Feldmann et al., OncoImmunology. 2020; Vol. 9, No. 1; Kittel-Boselli et al., Cancers 2021, 13, 4785; Daver et al., Leukemia 2021; 35:1843-1863; and Rotte et al., Immuno-Oncology Insights 2022; 3:13-24. Other than CAR-T cells, other types of engineered effector lymphocytes can also be employed in the practice of the methods of the invention, e.g., NK cells. See, e.g., Bachmann, Immunol. Letters 211:13-22, 2019; and Mitwasi et al., Sci. Rep. 10:2141, 2020. In addition to applications for cancer therapies, methods of the invention can also be employed in the treatment of various other diseases or conditions. These include, e.g., autoimmune diseases and infections (viral or fungal infections).

In addition to eliminating the chemotherapy conditioning that is indispensable in conventional CAR-T therapies, another major benefit achieved with the methods of the invention is a substantially reduced CAR-T cell dose that is required for administering to (e.g., infusion into) the subject for effective treatment or for in vivo expansion of the CAR-T cells. With conventional CAR-T therapies, typically a CAR-T cell dose of 1-2×10⁶ cells per kg of body weight is required. Such a high cell dose is exemplified by the two CD19 targeting CAR-T therapies approved by the FDA, Kymriah™ and Yescarta™. The product inserts of these two therapies both indicate such a recommended range of cell doses. In contrast, the methods of the present invention require a CAR-T cell dose that is typically less than about 1×10⁵ cells/kg. In some embodiments, the administered dose can be less than about 7.5×10⁴, 5×10⁴, 2.5×10⁴, 1×10⁴, 7.5×10³, 5×10³, 2.5×10³, 1×10³, 750, 500, 400, 300, 250, 200, 150, 100 cells per kg of body weight.

By eliminating the chemotherapy preconditioning treatment and also utilizing a substantially low CAR-T cell dose, the switchable CAR-T treatment methods of the invention provides a number of advantages over the conventional CAR-T treatment. Without the LD chemotherapy requirement, methods of the invention enable a reduction of the rate of infection. It is well known that LD chemotherapy leaves patients vulnerable to infections. Up to 50% or more of patients may experience infections after LD chemotherapy, resulting in their inability to receive subsequent CAR-T cell therapy, or in some cases death. Thus, the novel CAR-T therapies of the invention have a major advantage clinically. Additionally, elimination of LD preconditioning also reduces the time the patient needs to spend in the hospital, preserves any existing tumor immunity that the patient has, reduces the overall complexity of the treatment paradigm, and reduces the cost of the therapy.

Similarly, the need to use a substantially lower dose cell requirement also provides several advantages relative to conventional CAR-T therapies. These include, e.g., a reduction of the time, complexity, and cost of manufacturing. In addition, this leads to an increase in the ability to provide a purer cell product. A primary cause of failure in the manufacturing of conventional CAR-T cell products is the inability for the cell product to expand ex vivo (in culture) to sufficient cell numbers to meet dose requirement. The reduction in the total number of required cells could increase the percentage of cell manufactured lots that pass release specifications (i.e., lots would not fail due to insufficient cell numbers). Also, the therapies of the invention reduce potential adverse events related to infusion of the cells. Further, a lower cell dose also opens the possibility of higher purity cell products because cells can be purified to a higher degree. As high stringency of purification would result in cell loss, most manufacturing protocols do not use high stringency to avoid cell loss. When cell loss is not an issue because required cell numbers are very low, as with methods of the invention, the stringency of cell purification can be increased. Moreover, the low dose requirement also reduces the number of cells required to be collected from the patients.

### IV. CAR-T Switches and Complementary CAR-T Cells to be Used

The switchable CAR-T cell platform to be used in the methods of the invention contains one or more CAR-T switch molecules and one or more complementary CAR-T cells. In some embodiments, the administered one or more switches are complementary to the same CAR-T cell. Typically, the CAR-T switch to be administered to the subject contains a chimeric antigen receptor interacting domain (CAR-ID) and a targeting domain or targeting moiety. The CAR-ID specifically binds to the extracellular domain of the CAR on the complementary CAR-T cell. The CAR-ID of the CAR-T switches can be any substance that may be fused, conjugated, or otherwise attached to a targeting moiety described herein (e.g., an anti-CD19 antibody or antigen binding portion thereof), such that the CAR-ID is capable of being bound by the CAR of the CAR-T cells. For example, the CAR-ID may be a CAR-binding protein, a CAR-binding peptide, a CAR-binding small molecule. Other than the specific switches and CAR-T cells exemplified herein, methods of the invention can also be employed in other switchable CAR-T platforms known in the art. These include, as noted above, the UniCAR-T, RevCAR-T and ddBCMA-CAR-T platforms.

In some preferred embodiments, the CAR-ID contains a yeast transcription factor GCN4 peptide or a derivative or a homolog thereof. See, e.g., Hinnebusch and Fink, Proc Natl Acad Sci USA 80:5374-8, 1983; Arndt et al., Proc Natl Acad Sci USA 83:8516-20, 1986; WO2015057834 and WO2015057852. In some of these embodiments, the yeast transcription factor GCN4 peptide contains a GCN4 (7P14P) peptide sequence or epitope as described in Berger et al. FEBS Letters 450:149-153, 199; and Zahnd, C., et al., J. Biol. Chem. 279:18870-18877, 2004. As exemplification, the GCN4 derived peptide in the CAR-ID can contain the sequence NYHLENEVARLKKL (SEQ ID NO:1) or RMKQLEPKVEELLPKNYHLENEVARLKKLVGER (SEQ ID NO:13).

The targeting moiety of the employed CAR-T switch can bind to any target molecule that is present on the surface of a target tumor cell, e.g., CD19 as exemplified herein. Preferably, the target molecule can contain an antigen. In various embodiments, the target molecule can be a protein, a lipid moiety, a glycoprotein, a glycolipid, a carbohydrate, a polysaccharide, a nucleic acid, an MHC-bound peptide, or a combination thereof. In some preferred embodiments, the targeting moiety is a targeting polypeptide such as a targeting antibody or antigen-binding fragment thereof (e.g., an Fab). The targeting antibody can be human, fully human, humanized, human engineered, non-human, and/or chimeric antibody. In some embodiments, a non-human antibody to be used in the CAR-T switch can be humanized to reduce immunogenicity to humans, while retaining the specificity and affinity of the parental non-human antibody. In various embodiments, the targeting antibody can specifically bind to different target molecules on tumor cells, e.g., CD19, Her2, CLL1, CD33, CD123, EGFR, EGFRvIII, CD20, CD22, CS1, BCMA, CEA or a fragment thereof.

In some preferred embodiments, the targeting moiety of the CAR-T switch is an antibody or antigen-binding fragment that contains humanized heavy and/or light chain sequences. Some specific examples of tumor-targeting CAR-T switches that can be used and/or humanized for use in the methods of the invention are described, e.g., in PCT/US2017/057460, PCT/US2014/060713, PCT/US2014/060684, PCT/US2016/024524, PCT/US2016/027997, and PCT/US2016/027990.

In some embodiments, the CAR-ID and the targeting moiety are fused together. In some of these embodiments, a structural component (e.g., a peptide terminus) of the CAR-ID is joined with or liked to a terminus of a polypeptide targeting moiety (e.g., a humanized anti-CD19 antibody or an antigen binding fragment thereof). In some embodiments, the CAR-ID and the targeting moiety are fused together via a linker. In some embodiments, the CAR-ID is attached to the targeting moiety in a site-specific manner. Attachment in a site-specific manner may entail attaching the CAR-ID to a predetermined site on the targeting moiety. In some embodiments, site-specific attachment can entail attaching the CAR-ID to an unnatural amino acid in the targeting moiety.

Along with the CAR-T switch, a complementary CAR-T cell is also administered to a subject for the treatment of a cancer. Any CAR-T cell that can be bound by the employed CAR-T switch can be used in the methods of the invention. Typically, the CAR-T cells contain a chimeric antigen receptor (CAR) that contains an extracellular domain, a transmembrane domain and an intracellular signaling domain. The extracellular domain is capable of specifically binding to the CAR-ID (e.g., a GCN4, Flag, K4, or E4 peptide, or a small molecule such as FITC) of the employed CAR-T switch. In some preferred embodiments, the extracellular domain of the CAR contains an antibody or antibody fragment (e.g., a scFv) that binds to the CAR-ID of the switch. The CAR-antibody may be human, fully human, humanized, human engineered, non-human, and/or chimeric antibody. A number of known protein transmembrane domains can be used in the CAR of the CAR-T cells. In some embodiments, the transmembrane domain can be the transmembrane domain of CD8 or CD28. In general, the intracellular signaling domain can contain signaling domains such as CD3ζ, FcR-γ, and Syk-PT as well as co-signaling domains such as CD28, 4-1BB, and CD134. In some embodiments, the intracellular signaling domain of the CAR can contain (a) a CD3-zeta domain, plus (b) a CD28 domain, a 4-1BB domain, or both a CD28 domain and a 4-1BB domain. In some embodiments, a hinge region is present in the CAR to connect the extracellular domain with the transmembrane domain.

Depending on the CAR-ID in the employed CAR-T switch, various CAR-ID binding moieties can be present in the extracellular domains of the complementary CAR-T cells. In some embodiments, the extracellular domain of the CAR of the CAR-T cells can contain an anti-fluorescein isothiocyanate (FITC) antibody or a FITC-binding portion thereof. The anti-FITC antibody can be an anti-FITC scFv. In some embodiments, the extracellular domain of the CAR of the CAR-T cells can contain an antibody or fragment that recognizes a synthetic (non-naturally-occurring) peptide. For example, the antibody in the CAR can be one that specifically recognizes a FLAG® tag or a fragment thereof. In some embodiments, the extracellular domain of the CAR can contain an antibody or antibody fragment that recognizes a yeast transcription factor GCN4 or a fragment thereof. See, e.g., Rodgers et al., Proc Natl Acad Sci USA 113:E459-E468, 2016. In some embodiments, the extracellular domain of the CAR can contain an anti-HTP antibody or a fragment thereof.

When treating a human subject, the CAR of the administered CAR-T cells should preferably be human or humanized to reduce immunogenicity to humans. In some embodiments, the extracellular domain of the CAR of the administered CAR-T cells contain a humanized scFv that binds to the CAR-ID of the co-administered switch. The humanized scFv can comprise a humanized VH (variable heavy chain) sequence with non-human (e.g., murine) CDRs grafted onto a human immunoglobulin framework. In some embodiments, the extracellular domain of the CAR can contain a humanized anti-GCN4 scFv. Such scFv molecules can be derived from the anti-GCN4 scFv clone 52SR4 described in Zahnd et al., J. Biol. Chem. 279:18870-77, 2004. The humanized anti-GCN4 scFv (e.g., a humanized version of clone 52SR4) can contain a humanized light chain, a humanized heavy chain, or a humanized light chain and a humanized heavy chain. The humanized anti-GCN4 (e.g., a humanized version of clone 52SR4) can contain a humanized VH (variable heavy chain) sequence with non-human (e.g., murine) CDRs transplanted onto a human immunoglobulin framework. A specific example of such humanized anti-GCN4 scFv molecules that can be used in the CAR-T cells of the invention contains the sequence set forth in SEQ ID NO:7.

The complementary and inert CAR-T cells to be used in the invention can be prepared in accordance with methods well known in the art or specific protocols exemplified in, e.g., WO2018/075807, WO2015057834, WO2015057852, and Marcu-Malina et al., Expert Opinion on Biological Therapy, Vol. 9, No. 5. In general, recombinant technology can be used to introduce CAR-encoding genetic material into any suitable T-cells, e.g., human T cells such as central memory T-cells. As exemplification, the CAR-T cells to be used in the methods of the invention can be generated by transduction of human T cells with a lentiviral vector expressing the engineered CAR (e.g., a humanized CAR). A number of humanized CAR sequences and CAR-T cells harboring such humanized CAR sequences are known in the art. They can all be readily employed and/or adapted for use in the methods of the invention. See, e.g., WO2018/075807; Li et al., Biomarker Research 8:36, 2020; and Maude et al., Blood 128:217, 2016.

As a specific exemplification, the CAR-T switch to be used in the methods of the invention can contain a humanized anti-CD19 antibody or antigen-binding fragment thereof (e.g., a Fab) that is fused to a GCN4 peptide. The humanized anti-CD19 antibody can contain a light chain variable region sequence and a heavy chain variable region sequence that are identical to or substantially identical to (e.g., at least 95%, 96%, 97%, 98% or 99%) to SEQ ID NOs: 2 and 3, respectively. The GCN4 peptide CAR-ID can contain an amino acid sequence that is identical to or substantially identical to (e.g., at least 95%, 96%, 97%, 98% or 99%) to SEQ ID NO:1, and is fused to the anti-CD19 antibody at the N-terminus of the light chain. The complementary CAR-T cells to be employed along with this CAR-T switch contain in its extracellular domain a humanized anti-GCN4 antibody or antigen-binding fragment thereof (e.g., a scFv). The light chain variable region sequence and heavy chain variable region sequence of the anti-GCN4 antibody are shown in SEQ ID NOs: 4 and 5, respectively. A specific anti-GCN4 scFv molecule that has these two variable region sequences connected by a GS linker is shown in SEQ ID NO:7. In this CAR, a hinge region (SEQ ID NO:8) is present to connect the C-terminus of the scFv to the N-terminus of the transmembrane domain (SEQ ID NO:9). The intracellular domain of the CAR contains from the N-terminus to the C-terminus a CD28 domain (SEQ ID NO:10), a 4-1BB domain (SEQ ID NO: 11) and a CD3-zeta domain (SEQ ID NO:12). The amino acid sequence of the entire CAR molecule is shown in SEQ ID NO:6. Synthesis and production of this CD-19 targeting humanized CAR-T switch and the complementary CAR-T cells are described in the art. Many other humanized CAR-T switches and complementary CAR-T cells suitable for use in the methods of the invention are also known. See, e.g., WO18/075807; and Viaud et al., Proc. Natl. Acad Sci. USA 115:E10898-E10906, 2018.

(humanized anti-CD19 light chain variable region)
SEQ ID NO: 2
NYHLENEVARLKKLGGGGSDIQMTQSPSSLSASVGDRVTITCRASQDISK
YLNWYQQKPGKAVKLLIYHTSRLHSGVPSRFSGSGSGTDYTLTISSLQPE
DFATYFCQQGATLPYTFGQGTKLEIK
(humanized anti-CD19 heavy chain variable region)
SEQ ID NO: 3
QVQLQESGPGLVKPSETLSVTCTVSGVSLPDYGVSWIRQPPGKGLEWLGV
IWGSETTYYNSALKSRLTISKDNSKNQVSLKMSSLTAADTAVYYCAKHYY
YGGSYAMDYWGQGTLVTVSS
(humanized anti-GCN4 light chain variable region)
SEQ ID NO: 4
QAVVTQEPSLTVSPGGTVTLTCGSSTGAVTTSNYASWVQEKPDHLFRGLI
GGTNNRAPGVPARFSGSLLGGKAALTISGAQPEDEAIYFCVLWYSDHWVF
GGGTKLTVDG
(humanized anti-GCN4 heavy chain variable region)
SEQ ID NO: 5
QVQLQQSGPGLVKPSETLSITCTVSGFLLTDYGVNWVRQPPGKGLEWLGV
IWGDGITDYNPSLKSRLTVSKDTSKNQVSLKMSSLTDADTARYYCVTGLF
DYWGQGTTLTVSS
(full sequence of humanized CAR)
SEQ ID NO: 6
QAVVTQEPSLTVSPGGTVTLTCGSSTGAVTTSNYASWVQEKPDHLFRGLI
GGTNNRAPGVPARFSGSLLGGKAALTISGAQPEDEAIYFCVLWYSDHWVF
GGGTKLTVDGGGGGSGGGGSGGGGSGGGGSQVQLQQSGPGLVKPSETLSI
TCTVSGFLLTDYGVNWVRQPPGKGLEWLGVIWGDGITDYNPSLKSRLTVS
KDTSKNQVSLKMSSLTDADTARYYCVTGLFDYWGQGTTLTVSSESKYGPP
CPPCPDFWVLVVVGGVLACYSLLVTVAFIIFWVRSKRSRLLHSDYMNMTP
RRPGPTRKHYQPYAPPRDFAAYRSKRGRKKLLYIFKQPFMRPVQTTQEED
GCSCRFPEEEEGGCELRVKFSRSADAPAYKQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR
(humanized anti-GCN4 scFv)
SEQ ID NO: 7
QAVVTQEPSLTVSPGGTVTLTCGSSTGAVTTSNYASWVQEKPDHLFRGLI
GGTNNRAPGVPARFSGSLLGGKAALTISGAQPEDEAIYFCVLWYSDHWVF
GGGTKLTVDGGGGGSGGGGSGGGGSGGGGSQVQLQQSGPGLVKPSETLSI
TCTVSGFLLTDYGVNWVRQPPGKGLEWLGVIWGDGITDYNPSLKSRLTVS
KDTSKNQVSLKMSSLTDADTARYYCVTGLFDYWGQGTTLTVSS
(hinge of humanized CAR)
SEQ ID NO: 8
ESKYGPPCPPCPD
(transmembrane domain of humanized CAR)
SEQ ID NO: 9
FWVLVVVGGVLACYSLLVTVAFIIFWV
(CD28 domain of intracellular domain of humanized
CAR)
SEQ ID NO: 10
RSKRSRLLHSDYMNMTPRRPGPTRKHYQPYAPPRDFAAYRS
(4-1 BB domain of intracellular domain of
humanized CAR)
SEQ ID NO: 11
KRGRKKLLYIFKQPFMRPVQTTQEEDGCSCRFPEEEEGGCEL
(CD3-zeta domain of intracellular domain of
humanized CAR)
SEQ ID NO: 12
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

### V. Target Molecules and Tumors to be Treated

Some methods of the invention are directed to treating cancers by directing the CAR-T cells to tumor cells bearing one or more specific target molecules that are recognized by the co-administered CAR-T switches. The switches may interact with a plurality of target cells that express the same (e.g., CD19) or different target molecules (e.g., CD 19 and CD20). In some methods, the cancer cell surface molecule to be targeted by the sCAR-T platform of the invention can be a receptor. The receptor may be an extracellular receptor. The receptor may be a cell surface receptor. By way of non-limiting example, the receptor may bind a hormone, a neurotransmitter, a cytokine, a growth factor or a cell recognition molecule. The receptor may be a transmembrane receptor. The receptor may be an enzyme-linked receptor. The receptor may be a G-protein couple receptor (GPCR). The receptor may be a growth factor receptor. The cell surface molecule may be a non-receptor cell surface protein. The target molecule may be a cluster of differentiation proteins. By way of non-limiting example, the cell surface molecule may be selected from CD19, CD20, CD34, CD31, CD117, CD45, CD11b, CD15, CD24, CD114, CD182, CD14, CD11a, CD91, CD16, CD3, CD4, CD25, CD8, CD38, CD22, CD61, CD56, CD30, CD13, CLL1, CD33, CD123, or fragments or homologs thereof.

In addition to targeting the cancer markers noted above, methods of the invention can also target antigens or neoantigens that are presented by MHC I or MHC II molecules on the surface of tumor cells. In some preferred embodiments, these antigens are presented only by tumor cells and never by the normal ones. In some embodiments, the target antigens are tumor-specific antigens (TSAs) and, in general, result from a tumor-specific mutation. In some embodiments, the target antigens are antigens that are presented by tumor cells and normal cells, i.e., tumor-associated antigens (TAAs). Cytotoxic T lymphocytes that recognize these antigens may be able to destroy the tumor cells before they proliferate or metastasize. Tumor antigens may also be on the surface of the tumor in the form of, for example, a mutated receptor, in which case they may be recognized by B cells.

In some embodiments, the target molecule on the tumor cell surface can be a molecule that does not comprise a peptide. The cell surface molecule may comprise a lipid. The cell surface molecule may comprise a lipid moiety or a lipid group. The lipid moiety may comprise a sterol. The lipid moiety may comprise a fatty acid. The antigen may comprise a glycolipid. The cell surface molecule may comprise a carbohydrate.

In some exemplified embodiments, the CAR-T switch to be used in the methods of the invention contains a CAR-binding peptidyl antigen such as a peptide from a yeast transcription factor peptide and a targeting polypeptide. In some of these embodiments, the yeast transcription factor peptide is a GCN4 derived peptide (a GCN4 peptide), the targeting polypeptide contains a targeting antibody or antibody fragment, e.g., an anti-CD19 antibody or a fragment thereof. The targeting antibody or antibody fragment may comprise an anti-Her2 antibody or a fragment thereof. The targeting antibody or antibody fragment may be selected from an anti-CS1 antibody, an anti-BCMA antibody, an anti-EGFRvIII antibody, an anti-CD20 antibody, an anti-EGFR antibody, an anti-CEA antibody, an anti-CLL1 antibody, an anti-CD33 antibody, an anti CD123 antibody, and fragments thereof.

In some embodiments, the employed CAR-T switches contains a CAR binding hydrophilic target peptide (HTP) and a targeting polypeptide. In these embodiments, the targeting polypeptide can be an anti-CD19 antibody or a fragment thereof. In various embodiments, the targeting polypeptide can contain an anti-Her2 antibody, an anti-CS1 antibody, an anti-BCMA antibody, an anti-EGFRvIII antibody, an anti-CD20 antibody, an anti-EGFR antibody, an anti-CEA antibody, an anti-CLL1 antibody, an anti-CD33 antibody, or an anti CD123 antibody, or a fragment thereof.

Various cancers can be treated with the methods of the invention. These include cancers derived from any tissue such as, e.g., a tissue of a brain, an esophagus, a breast, a colon, a lung, a glia, an ovary, a uterus, a testicle, a prostate, a gastrointestinal tract, a bladder, a liver, a thyroid and skin. In some embodiments, the cancer to be treated is derived from bone. In some embodiments, the cancer to be treated is derived from blood. In these embodiments, the cancer can be derived from a B cell, a T cell, a monocyte, a thrombocyte, a leukocyte, a neutrophil, an eosinophil, a basophil, a lymphocyte, a hematopoietic stem cell or an endothelial cell progenitor. In some embodiments, the cancer can be derived from a CD19-positive B lymphocyte. In some embodiments, the cancer may be derived from a stem cell. For example, the targeting cancer cell may be derived from a pluripotent cell. In some embodiments, the cancer cell to be targeted can be derived from one or more endocrine glands. The endocrine gland may be a lymph gland, pituitary gland, thyroid gland, parathyroid gland, pancreas, gonad or pineal gland.

Many tumors or cell proliferative disorders can be treated with methods of the invention. These include solid tumors, lymphomas, leukemias and liposarcomas. The disorders to conditions to be treated can be acute, chronic, recurrent, refractory, accelerated, in remission, stage I, stage II, stage III, stage IV, juvenile or adult. Solid tumors that can be treated with methods of the invention include, e.g., cancers originated or derived from a brain, an esophagus, a breast, a colon, a lung, a glia, an ovary, a uterus, a testicle, a prostate, a gastrointestinal tract, a bladder, a liver, a thyroid and skin.

In some embodiments, the cancer to be treated is heterogeneous. In some embodiments, the cancer to be treated is a blood cell malignancy. For example, the cancer to be treated can be derived from bone marrow cells or other blood cells. In these embodiments, the cancer can be derived from a B cell, a T cell, a monocyte, a thrombocyte, a leukocyte, a neutrophil, an eosinophil, a basophil, a lymphocyte, a hematopoietic stem cell or an endothelial cell progenitor. In some embodiments, the cancer can be derived from a CD19-positive B lymphocyte. In some embodiments, the cancer may be derived from a stem cell. For example, the targeting cancer cell may be derived from a pluripotent cell. In some embodiments, the cancer cell to be targeted can be derived from one or more endocrine glands. The endocrine gland may be a lymph gland, pituitary gland, thyroid gland, parathyroid gland, pancreas, gonad or pineal gland.

In some embodiments, the cancer to be treated is a Her2-positive cancer. These include, e.g., Her2-positive breast cancer and Her2-positive pancreatic cancer. In some embodiments, the cancer to be treated can be a BCMA-positive cancer, e.g., a BCMA-positive multiple myeloma. In some embodiments, the cancer to be treated can be a CS1-positive cancer, e.g., a CS1-positive multiple myeloma. In some embodiments, the cancer to be treated can be a EGFRvIII-positive cancer cell, e.g., a EGFRvIII-positive glioblastoma. In some other embodiments, the cancer to be treated can be a CD20-positive cancer, a CD22-positive cancer, a CD123-positive cancer or a CD33-positive cancer. These include, e.g., CD33-positive acute myeloid leukemia and CD123-positive acute myeloid leukemia. In some embodiments, the cancer to be treated can be a CLL1-positive cancer, e.g., CLL1-positive acute myeloid leukemia.

Some methods of the invention are directed to treating leukemia, e.g., CD-19 positive leukemia. Specific examples of leukemias include myelogenous leukemia, lymphoblastic leukemia, myeloid leukemia, an acute myeloid leukemia, myelomonocytic leukemia, neutrophilic leukemia, myelodysplastic syndrome, B-cell lymphoma, Burkitt lymphoma, large cell lymphoma, mixed cell lymphoma, follicular lymphoma, mantle cell lymphoma, Hodgkin lymphoma, recurrent small lymphocytic lymphoma, hairy cell leukemia, multiple myeloma, basophilic leukemia, eosinophilic leukemia, megakaryoblastic leukemia, monoblastic leukemia, monocytic leukemia, erythroleukemia, erythroid leukemia and hepatocellular carcinoma. In some embodiments, the disorder to be treated is a hematological malignancy. In some embodiments, the disorder to be treated is a B cell malignancy. In some embodiments, the disorder to be treated is a chronic lymphocytic leukemia. In some embodiments, the disorder to be treated is an acute lymphoblastic leukemia. In some embodiments, the disorder to be treated is a CD19-positive Burkitt's lymphoma.

In some preferred embodiments, the cancer to be treated is a CD19-positive tumor or malignancy. In some of these embodiments, the cancer to be treated is a. In some of these embodiments, the cancer to be treated is a B cell cancer or B cell malignancy. B cell cancer or B cell malignancy encompass B-cell lymphomas which account for a major portion of non-Hodgkin lymphomas (NHL). Examples of these B cell cancers include, e.g., diffuse large B-cell lymphoma (DLBCL), follicular lymphoma, chronic lymphocytic leukemia (CLL), small lymphocytic lymphoma (SLL), mantle cell lymphoma (MCL), marginal zone lymphoma, Burkitt lymphoma, lymphoplasmacytic lymphoma (Waldenstrom macroglobulinemia), hairy cell leukemia (HCL), primary central nervous system (CNS) lymphoma, and primary intraocular lymphoma.

### VI. Administration and Dosages

In the practice of the therapeutic methods of the invention, subjects in need of treatment are administered a pharmaceutical composition that contains a therapeutically effective amount of the sCAR-T platform described herein. In addition to the sCAR-T platform, the composition can contain one or more pharmaceutically acceptable carrier or agent, e.g., salts, excipients or vehicles. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The administration of the sCAR-T platform may be in any order. For example, in some embodiments, the CAR-T cells may be administered prior to CAR-T switch administration. In some embodiments, the CAR-T switch may be administered prior to CAR-T cell administration. In some embodiments, the CAR-T cells may be administered simultaneously with CAR-T switch administration.

General guidance for preparation and administration of the therapeutic compositions of the invention are described in the art. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999). Methods of administering therapeutic compositions to a subject can be accomplished based on procedures routinely practiced in the art. See, e.g., Remington: The Science and Practice of Pharmacy, Mack Publishing Co., 20^{th} ed., 2000; Iwasaki et al., Jpn. J. Cancer Res. 88:861-6, 1997; Jespersen et al., Eur. Heart J. 11:269-74, 1990; and Martens, Resuscitation 27:177, 1994. Typically, the sCAR-T platform-containing composition is administered (e.g., via injection) in a physiologically tolerable medium, such as phosphate buffered saline (PBS). The therapeutic compositions can be administered to subjects in need of treatment via any appropriate route. In some preferred embodiments, the composition is administered to the subject via parenteral administration. In these embodiments, the administered composition should contain a sterile aqueous preparation that is preferably isotonic with the blood of the recipient.

The sCAR-T switch and the complementary CAR-T cells should be administered to the subject in a sufficient amount to inhibit the growth and promote the regression of a tumor in the subject. As noted above, the number of CAR-T cells to be administered to a subject afflicted with a tumor is substantially lower than that used in conventional CAR-T therapies. Nonetheless, the low doses of CAR-T cells used in the methods of the invention should be sufficient for arresting tumor growth and/or for promoting tumor regression in the subject. The sufficient number of cells can be determined based on the type of tumor, its size and stage of development, the route of administration, the age of the specific subject to be treated and other factors that will be readily apparent to one of ordinary skill in the art of treating tumors. As a general guidance for administration via injection or implantation, the subject to be treated is administered with a single CAR-T cell dose containing from about 100 cells to about 1×10⁵ cells per kg of body weight. In various embodiments, the single dose can contain less than about 7.5×10⁴, 5×10⁴, 2.5×10⁴, 1×10⁴, 7.5×10³, 5×10³, 2.5×10³, 1×10³, 750, 500 or 300 cells per kg of body weight. For the CAR-T switch molecule, a single dose can be in the range between about 0.001 mg to about 10 mg per kg of body weight. In some embodiments, the single dose of the switch to be administered is between about 0.005 mg to about 2.5 mg per kg of body weight. In some embodiments, the single dose of the switch to be administered is any amount from about 0.0075 mg (or 0.01 mg) to about 0.5 mg (or 1 mg, 1.5 mg or 2 mg) per kg of body weight. In various embodiments, a single dose of the CAR-T switch molecule to be administered can be about 0.0025 mg, 0.005 mg, 0.0075 mg, 0.01 mg, 0.025 mg, 0.05 mg, 0.075 mg, 0.1 mg, 0.25 mg, 0.5 mg, 0.75 mg, 1 mg, 1.5 mg, 2 mg, 2.5 mg, 3 mg, 4 mg, 5 mg or more per kg of body weight.

In addition, the noted doses of CAR-T cells and the CAR-T switch molecule can be administered in a single administration or by multiple administration over a period of time, as determined by the clinician in charge of the treatment. In various embodiments, administration of the noted cell dose can be based on any frequency that is between daily to once every two years. Thus, depending on the attending circumstances (e.g., type and severity of the tumor to be treated, administration route, and age of the subject), the subject can receive the administration every day, every other day, twice per week, weekly, biweekly, monthly, every two months, every three months, every four months, every five months, every six months, or a longer interval.

In some embodiments, the CAR-T cells are administered to the subject to be treated only at the beginning of the treatment (e.g., once or twice administration), while the CAR-T switch molecule is administered multiple times based on an “on/off schedule” during the course of the treatment, e.g., throughout the first few weeks or months thereafter. As exemplified herein, the on/off schedule comprises multiple cycles, with each cycle containing an “on” phase and an “off” phase. The CAR-T switch is administered to the subject during the “on” phase, while no CAR-T switch is administered during the “off” phase. In various embodiments, the subject can be administered the switch molecule twice a day, daily, every other day, every three days or longer during an “on” phase, which lasts a specific period of time (e.g., any length from about one day to a few weeks). The “off” phase of the cycle can be any period that is longer than the interval of the administration during the “on” phase. Thus, for example, when the administration takes place every day or every other day during the “on” phase, the “off” phase can last any length of time from about a few days to about a few years. In various embodiments, the “on” phase can be, e.g., 1 day, 2 days, 3 days, 4 days, 5 days, 6 days, 1 week, 2 weeks, 3 weeks, or longer, with administration taking place once every day, every two days, every 3 days or longer. For each of these “on” phases, the “off” phase can be, e.g., 4 days, 5 days, 6 days, 1 week, 2 weeks, 3 weeks, 4 weeks, 2 months, 3 months, 4 months, 5 months, 6 months, 1 year or longer. The length of time of the “on” phase and the “off” phase can be respectively the same for the multiple cycles. Alternatively, the duration of the “on” phase and/or the “off” phase can vary among the different cycles.

The number of cells and frequency of administration can also vary depending on whether the treatment is prophylactic or therapeutic. In prophylactic applications, a relatively low number of cells may be administered at relatively infrequent intervals over a long period of time. Some subjects may continue to receive treatment for the rest of their lives. In therapeutic applications, a relatively high number of cells at relatively short intervals may be required until progression of the tumor is reduced or terminated, and preferably until the subject shows partial or complete regression of the tumor. Thereafter, the subject can be administered a prophylactic regime.

In some embodiments, therapeutic compositions described herein can be administered locally via implantation into the affected area of a membrane, sponge, or other appropriate material on to which a sCAR-T platform disclosed herein has been absorbed or encapsulated. Where an implantation device is used, the device may be implanted into any suitable tissue or organ, and delivery of a CAR-T platform disclosed herein may be directly through the device via bolus, or via continuous administration, or via catheter using continuous infusion.

As noted above, the sCAR-T platform described herein can be used in combination with other known regimens for treating cancers. Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

### EXAMPLES

The following examples are provided to further illustrate the invention but not to limit its scope. Other variants of the invention will be readily apparent to one of ordinary skill in the art and are encompassed by the appended claims.

### Example 1. No Preconditioning Required for sCAR-T Treatment Platform

### Model and Methods:

To challenge the notion that a successful CAR-T cells engraftment requires pre-conditioning, we transferred 3e6 murine sCAR-T cells and an anti-murine CD19 switch (based on the CAR-T platform described in Viaud et al., Proc. Natl. Acad Sci. USA 115: E10898-E10906, 2018) into tumor free Syngeneic B6 mice, using T-cells activated with soluble antiCD3 and antiCD28 antibodies or bead-conjugated CD3/CD28 antibodies. On day-1, mice were randomized, and IP injected with Cyclophosphamide (CTX) at, 100 mg-Bead activation (D), 100 mg-Soluble activation (E), 50 mg-Soluble activation (F), 0 mg/kg-Soluble activation (G) and 0 mg/kg Bead-activation (H), plus appropriate controls groups for the different CTX doses (A-C). The next day, CAR-T cells were transferred, and 4 hrs. later, switch was administered for 1 week using four 1 mg/kg switch doses q.o.d. then switch doses were paused for 3 weeks to complete a treatment cycle. In total, five consecutive treatment cycles were performed with a sixth one administered after 16 weeks pause, except for group (H) that did not receive the sixth cycle. At designated intervals, mice were bled, and the activity of the CAR-T was monitored indirectly via the level of CD19+ B cell depletion by flow cytometry.

### Study Results:

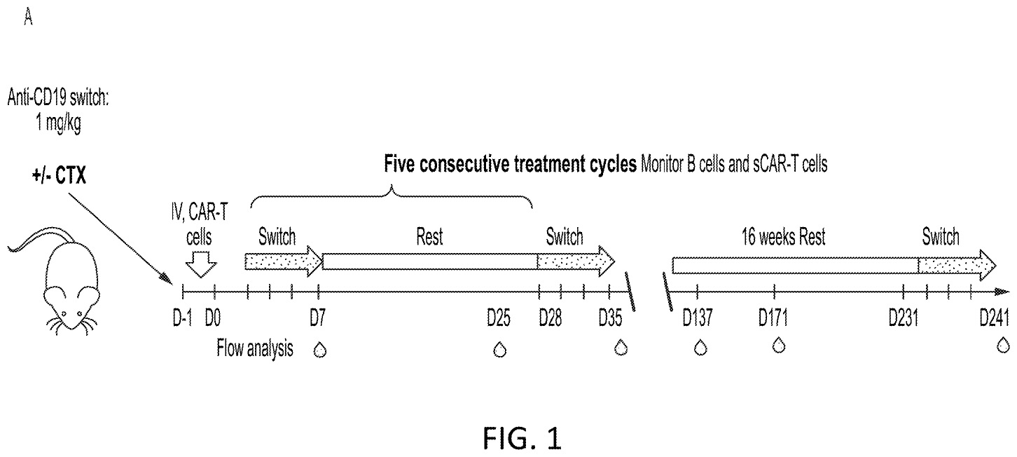

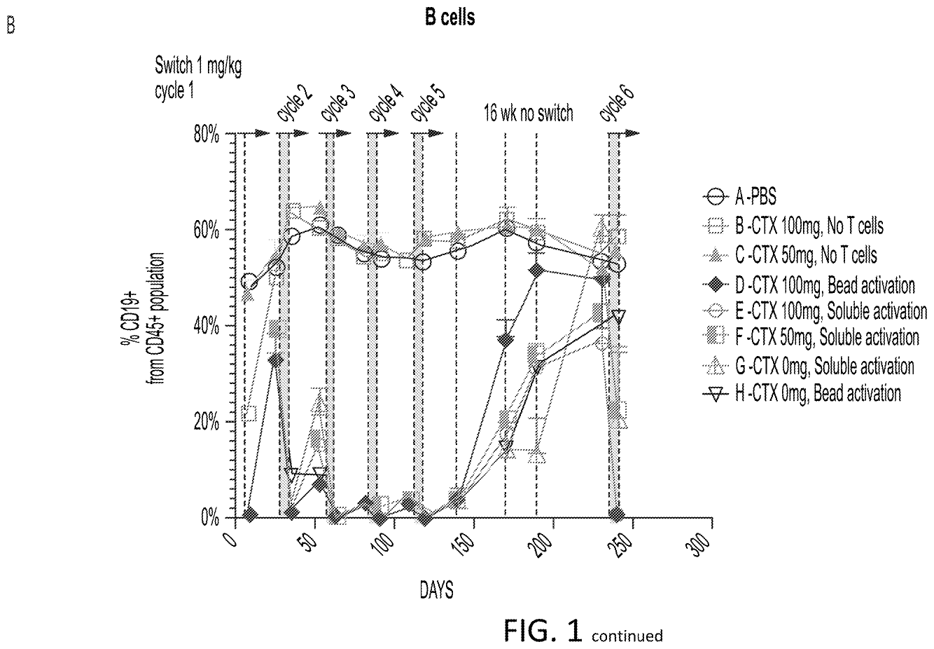

FIG. 1 shows near complete B cell depletion in peripheral blood in all cohorts treated with switch at the end of cycle 1, regardless of the cyclophosphamide dose used 100 mg, 50 mg as well as in the groups that do not receive any preconditioning. The activation method used to manufacture the CAR-T cells played no role in the successful engraftment of the sCAR-T cells, demonstrating that switchable CAR-T cells may use a variety of manufacturing methods to facilitate engraftment and activity in an immunocompetent host. Furthermore, after depletion during switch administration, B cells were again detected in the periods in which switch was not dosed, but with each successive treatment cycle, the relative percentage of B cells decreased in comparison to the antecedent cycle as the depletion of the B cells become more pervasive in response to the expanding CART-cells. However, B cell returned to physiological pre-treatment levels after a 16 wks of switch pause.

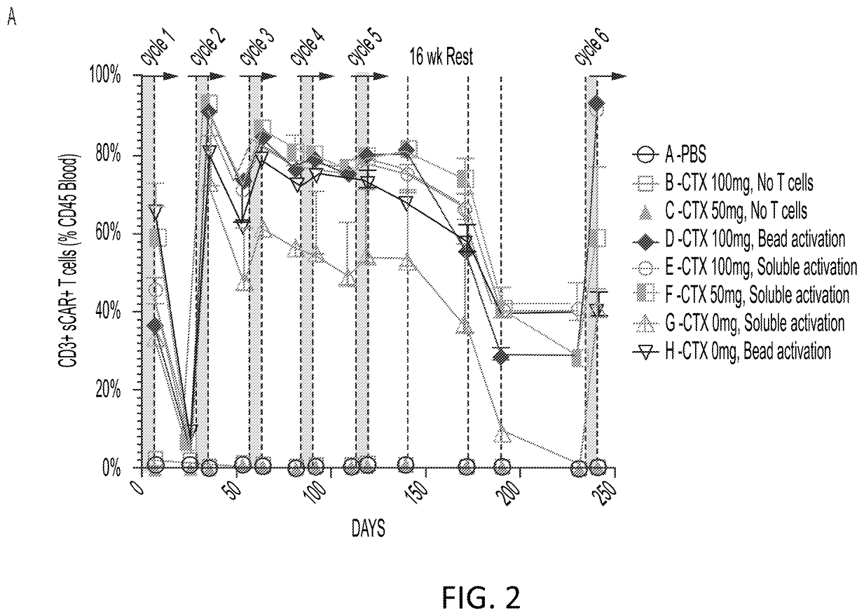

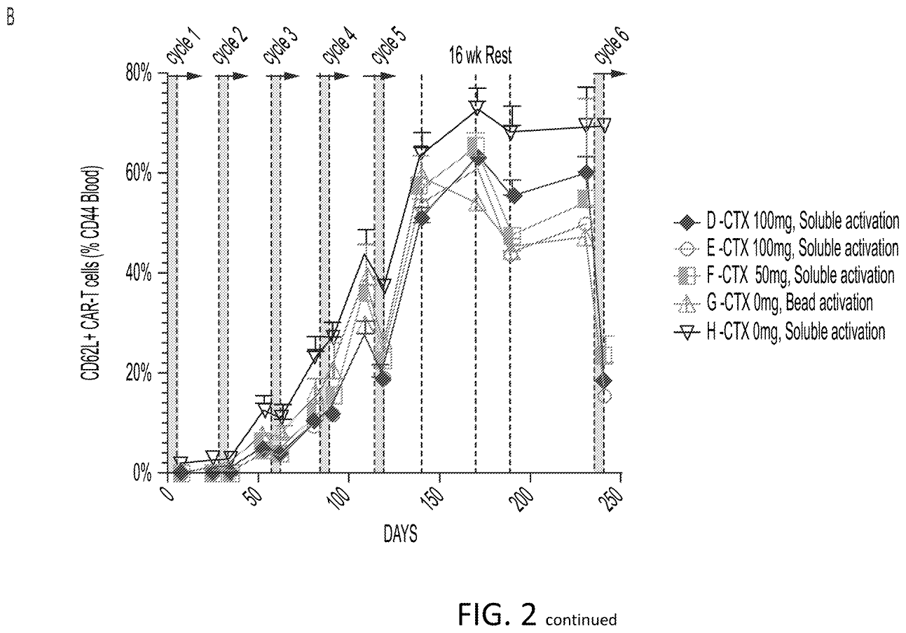

FIG. 2, Panel A, demonstrates that the treatment cycles consisting of a switch treatment for 1 week using four 1 mg/kg switch doses q.o.d. and then pausing for 3 weeks elicits in vivo CAR-T cell expansion, as the 3-week rest period enables the sCAR-T cells to “rest” It also allows B cell repopulation which is important as the B cells act as self-prime-boost for CAR-T expansion. By the second cycle, sCAR-T+ cells become the predominant T-cell population, representing close to 70% of all CD3⁺ cells in circulation. This sCAR+ Population remained very stable during contraction even after 16 weeks and re-expanded once more when re-call, after more than a hundred days with no stimulation. To emphasize that the expansion is switch dependent, the sixth switch cycle was withheld to group (H) there was no re-expansion.

FIG. 2, Panel B, indicates that progressive accumulation of CD44+/CD62L+ (memory) sCAR-T cells may explain the remarkable CAR-T cell's persistence in the model. Even after 16 weeks and re-expanded when re-call, after over a hundred days with no stimulation . . . .

### Example 2. sCAR-T Engraftment and B Cell Depletion with Fewer sCAR-T Cells

### Model and Methods:

As a follow-up, and to determine the lower limit of detectable sCAR-T cell engraftment we could accomplish., A titrated number of sCART-cells were transferred, beginning with 3e6 as in our previous experiment and down a 10-fold dilution. As previously described, murine sCAR-T cells were transferred, and anti-murine CD19switch was administered into tumor-free Syngeneic B6 mice, using t-cells activated with bead-conjugated CD3/CD28 antibodies. On day 0, mice were randomized, and CAR-T cells were transferred, 4 hrs. later switch was administered for 1 week using four 1 mg/kg switch doses q.o.d. and then the witch dosing was held for 3 weeks to complete a treatment cycle. A total of 5 consecutive treatment cycles were performed, mice were bled, and the activity of the CAR-T was assessed via the level of CD19⁺ B cell depletion by flow cytometry.

### Study Results:

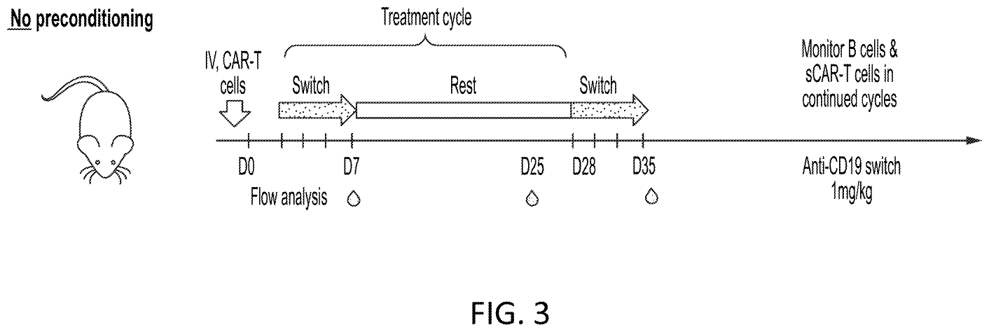

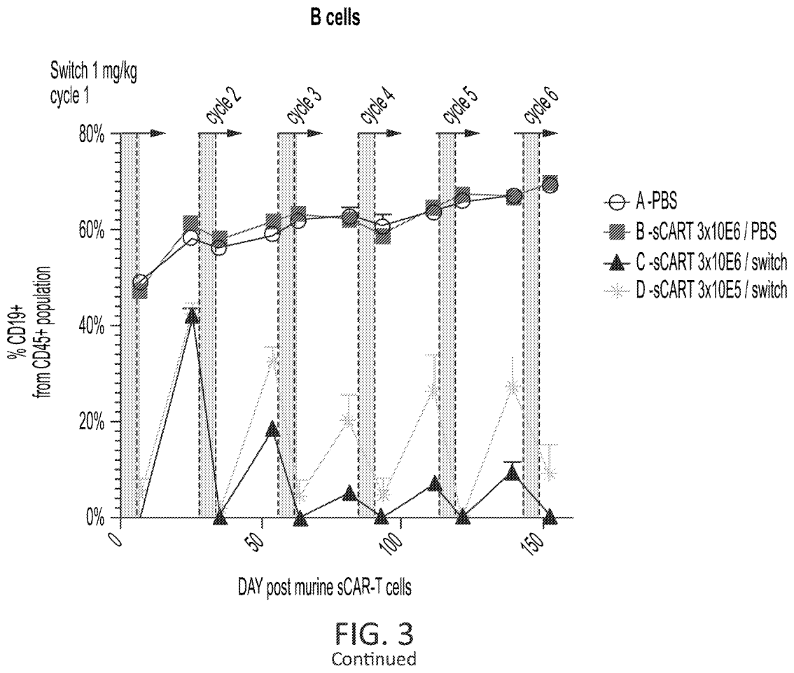

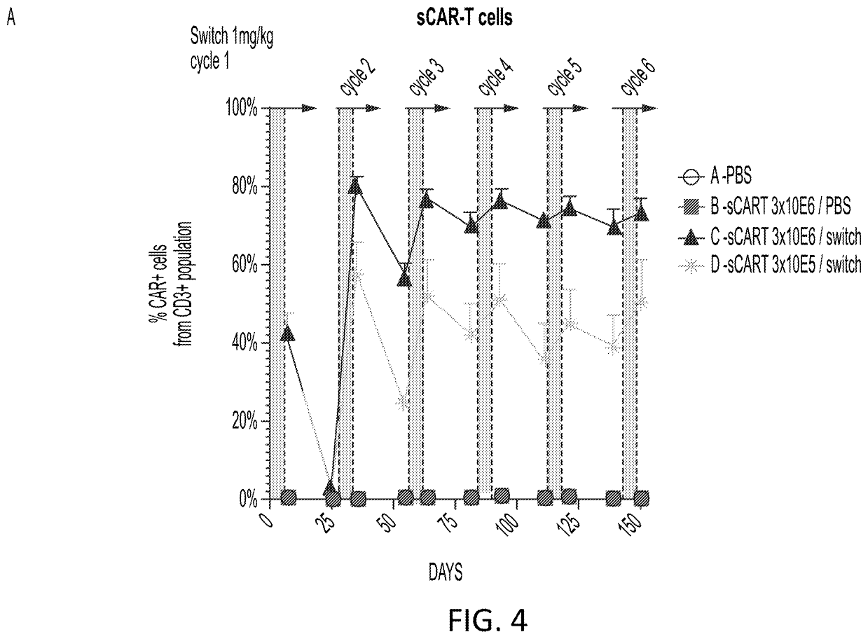

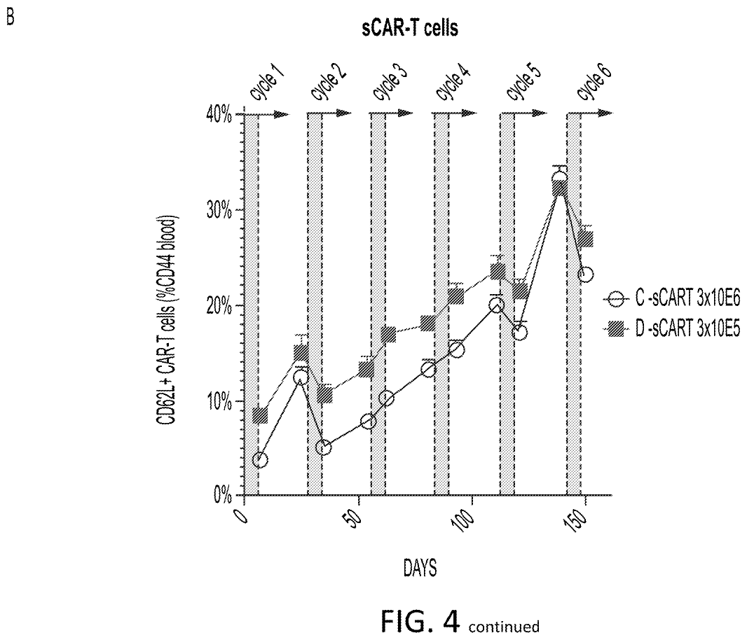

As in our previous experiment, there was a near-complete B cell depletion at the end of the first switch dosing period in mice treated at 3e6 sCAR-T cells per mouse and mice treated with a tenfold lower concentration of 3e5 CAR-T cell per mouse (FIG. 3). FIG. 4 shows that on/off cycling led to an important CAR-T cell expansion as described in our previous experiment. sCAR-T cells were detectable by flow cytometry at the dose of 3×10⁶ per mouse and a tenfold lower dose at 3×10⁵ per mouse. The results show the progressive accumulation of CART cells in the blood (FIG. 4, Panel A) and CD44+/CD62L+ (memory) sCAR-T over time (FIG. 4, Panel B). These results indicate that even with a tenfold lower dilution the sCAR-T platform, the treatment does not require preconditioning for engraftment.

### Example 3. Comparison of Potency Between sCAR-T and Conventional CAR-T

### Model and Methods:

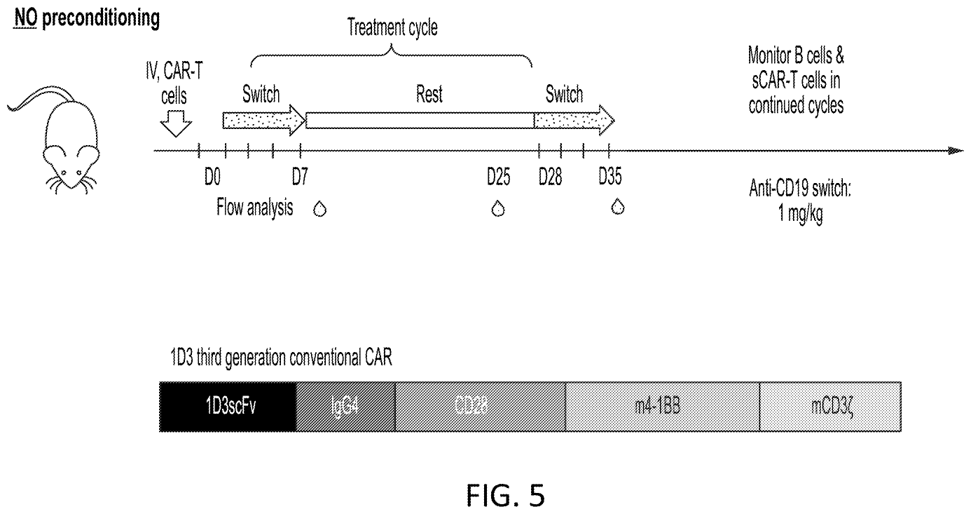

To compare the potency of our switchable CAR platform to that of a comparable conventional CAR. a fully murine third-generation CAR construct was generated, compose of the 1D3 Rat anti-mouse CD19 scFv, an IgG4 extracellular spacer followed by CD28, 41BB, and CD3z (FIG. 5). After testing the new construct activity in vitro, we followed a similar experiment and transferred 3e6 1D3-Conventional CAR-T cells per mouse and compare These mice with a cohort of mice dosed with switchable sCAR-T cells at a dose of 3e5 per mouse, the lowest dose of sCAR-T cell that we could detect in peripheral blood in our previous experiment. Otherwise, we used the same t-cell activation protocol as well as treatment cycle structure previously described.

### Study Results:

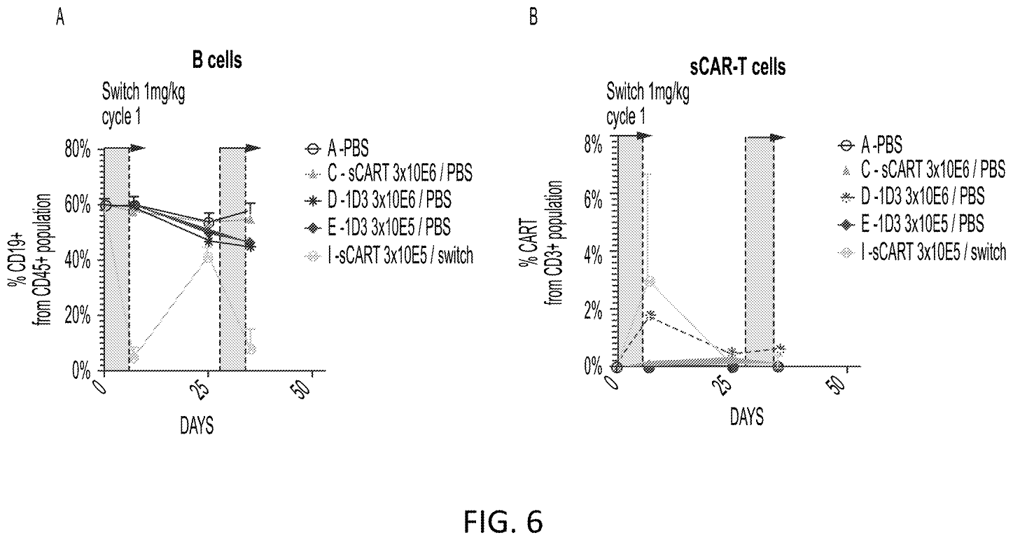

The CD19⁺ cell population reminded unaffected in mice groups treated with the 1D3 conventional CAR, as shown in FIG. 6, Panel A, even though conventional CAR-T cells were detectable at the end of the week mark (FIG. 6, Panel B). This highlights the notion that conventional CAR-T cells require preconditioning to be effective as widely accepted (see, e.g., Gattinoni et al., J. Exp. Med. 2005; 202:907-912; and Klebanoff et al., Trends Immunol. 2005; 26:111-117). In contrast, there was a near complete depletion of CD19⁺ cells in the Switchable CAR-T cell group treated at 3×10⁵ cells, CART-cell are at least 10,000-fold more potent that of conventional CAR-T cells.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

1. A method of engrafting or expanding an engineered T cell in a subject in need thereof, comprising contacting the subject with (a) a chimeric antigen receptor-T cell switch molecule (CAR-T switch) comprising (i) a chimeric antigen receptor-interacting domain (CAR-ID) and (ii) a targeting moiety, and (b) a complementary CAR-T cell comprising in the extracellular domain of its CAR a single-chain variable fragment (scFv) that specifically binds to the CAR-ID; wherein the subject has not undergone lymphodepletion (LD) chemotherapy prior to being administered the CAR-T switch and the CAR-T cell; thereby engrafting or expanding the engineered T cell in the subject.

2. The method of claim 1, wherein the targeting moiety specifically binds to a cell surface target molecule in the subject.

3. The method of claim 1, wherein the targeting moiety comprises an antibody moiety or a small organic molecule.

4. The method of claim 1, wherein the target molecule is present on the surface of a cell associated with or implicated in a disease in the subject.

5. (canceled)

6. A method of treating, or stopping or slowing the development of, a disease in a subject, comprising administering to the subject (a) a first chimeric antigen receptor-T cell switch molecule (CAR-T switch) comprising (i) a chimeric antigen receptor-interacting domain (CAR-ID) and (ii) a targeting moiety that specifically binds to target molecule that is present on the surface of a cell associated with or implicated in the disease, and (b) a complementary CAR-T cell comprising in the extracellular domain of its CAR a single-chain variable fragment (scFv) that specifically binds to the CAR-ID; wherein the targeting moiety specifically binds to a cell surface molecule in the subject; and wherein the subject has not undergone lymphodepletion (LD) chemotherapy prior to being administered the CAR-T switch and the CAR-T cell; thereby treating, stopping or slowing the development of, the disease in the subject.

7. The method of claim 6, wherein the subject is a human.

8. The method of claim 6, wherein the disease is a cancer or an autoimmune disease.

9. The method of claim 6, wherein the targeting moiety is an antibody moiety or a small molecule moiety.

10. The method of claim 6, wherein the subject is administered with one dose of the CAR-T cell at the beginning of the treatment, and multiple doses of the CAR-T switch during the course of the treatment.

11. The method of claim 10, wherein a single dose of the CAR-T cells administered to the subject is less than about 1×10⁵ cells per kg of body weight.

12. The method of claim 10, wherein a single dose of CAR-T cells administered to the subject is less than about 7.5×10⁴, 5×10⁴, 2.5×10⁴, 1×10⁴, 7.5×10³, 5×10³, 2.5×10³, 1×10³, 750, or 500 cells per kg of body weight.

13. The method of claim 10, wherein a single dose of the CAR-T switch administered to the subject is from about 0.01 mg to about 1 mg per kg of body weight.

14. (canceled)

15. The method of claim 6, wherein the CAR-ID comprises a GCN4 derivative peptide, and the CAR extracellular domain comprises an anti-GCN4 scFv.

16. The method of claim 15, wherein the GCN4 derivative peptide comprises SEQ ID NO: 1, a substantially identical sequence or a conservatively modified variant thereof.

17. The method of claim 15, wherein the anti-GCN4 scFv comprises light chain variable region sequence set forth in SEQ ID NO:4, and heavy chain variable region sequence set forth in SEQ ID NO:5.

18. The method of claim 15, wherein the anti-GCN4 scFv comprises the amino acid sequence SEQ ID NO:7.

19. (canceled)

20. (canceled)

21. The method of claim 6, wherein the targeting moiety is an anti-CD19 antibody or antigen-binding fragment thereof that comprises a light chain variable region and a heavy chain variable region sequence set forth as SEQ ID NOs: 2 and 3, respectively.

22. (canceled)

23. (canceled)

24. (canceled)

25. (canceled)

26. The method of claim 6, wherein the CAR of the CAR-T cell is humanized and comprises the amino acid sequence SEQ ID NO:6.

27. (canceled)

28. The method of claim 6, wherein the cancer to be treated is a CD19-positive malignancy.

29. The method of claim 28, wherein the CD19-positive malignancy is acute lymphoblastic leukemia, acute myeloid leukemia, or chronic lymphocytic leukemia.

30. (canceled)

31. (canceled)


SEQUENCE LISTING (13 sequences)

SEQ ID NO: 1 - protein, 14 aa, Saccharomyces cerevisiae
NYHLENEVARLKKL

SEQ ID NO: 2 - protein, 126 aa, Homo sapiens
NYHLENEVARLKKLGGGGSDIQMTQSPSSLSASVGDRVTITCRASQDISKYLNWYQQKPGKAVKLLIYHTSRLHSGVPSRFSGSGSGTDYTLTISSLQPEDFATYFCQQGATLPYTFGQGTKLEIK

SEQ ID NO: 3 - protein, 120 aa, Homo sapiens
QVQLQESGPGLVKPSETLSVTCTVSGVSLPDYGVSWIRQPPGKGLEWLGVIWGSETTYYNSALKSRLTISKDNSKNQVSLKMSSLTAADTAVYYCAKHYYYGGSYAMDYWGQGTLVTVSS

SEQ ID NO: 4 - protein, 110 aa, Homo sapiens
QAVVTQEPSLTVSPGGTVTLTCGSSTGAVTTSNYASWVQEKPDHLFRGLIGGTNNRAPGVPARFSGSLLGGKAALTISGAQPEDEAIYFCVLWYSDHWVFGGGTKLTVDG

SEQ ID NO: 5 - protein, 113 aa, Homo sapiens
QVQLQQSGPGLVKPSETLSITCTVSGFLLTDYGVNWVRQPPGKGLEWLGVIWGDGITDYNPSLKSRLTVSKDTSKNQVSLKMSSLTDADTARYYCVTGLFDYWGQGTTLTVSS

SEQ ID NO: 6 - protein, 478 aa, Homo sapiens
QAVVTQEPSLTVSPGGTVTLTCGSSTGAVTTSNYASWVQEKPDHLFRGLIGGTNNRAPGVPARFSGSLLGGKAALTISGAQPEDEAIYFCVLWYSDHWVFGGGTKLTVDGGGGGSGGGGSGGGGSGGGGSQVQLQQSGPGLVKPSETLSITCTVSGFLLTDYGVNWVRQPPGKGLEWLGVIWGDGITDYNPSLKSRLTVSKDTSKNQVSLKMSSLTDADTARYYCVTGLFDYWGQGTTLTVSSESKYGPPCPPCPDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 243 aa, Homo sapiens
QAVVTQEPSLTVSPGGTVTLTCGSSTGAVTTSNYASWVQEKPDHLFRGLIGGTNNRAPGVPARFSGSLLGGKAALTISGAQPEDEAIYFCVLWYSDHWVFGGGTKLTVDGGGGGSGGGGSGGGGSGGGGSQVQLQQSGPGLVKPSETLSITCTVSGFLLTDYGVNWVRQPPGKGLEWLGVIWGDGITDYNPSLKSRLTVSKDTSKNQVSLKMSSLTDADTARYYCVTGLFDYWGQGTTLTVSS

SEQ ID NO: 8 - protein, 13 aa, Artificial Sequence
  synthetic sequence
ESKYGPPCPPCPD

SEQ ID NO: 9 - protein, 27 aa, Artificial Sequence
  Synthetic sequence
FWVLVVVGGVLACYSLLVTVAFIIFWV

SEQ ID NO: 10 - protein, 41 aa, Artificial Sequence
  Synthetic sequence
RSKRSRLLHSDYMNMTPRRPGPTRKHYQPYAPPRDFAAYRS

SEQ ID NO: 11 - protein, 42 aa, Artificial Sequence
  Synthetic sequence
KRGRKKLLYIFKQPFMRPVQTTQEEDGCSCRFPEEEEGGCEL

SEQ ID NO: 12 - protein, 112 aa, Artificial Sequence
  Synthetic sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 33 aa, Saccharomyces cerevisiae
RMKQLEPKVEELLPKNYHLENEVARLKKLVGER